(19) United States
(12) Patent Application Publication
(10) Pub. No.: US 2009/0291150 A1
(43) Pub. Date: Nov. 26, 2009

(54) METHOD AND COMPOSITIONS FOR TREATING AND PREVENTING SEIZURES BY MODULATING ACID-SENSING ION CHANNEL ACTIVITY

(75) Inventors: MICHAEL J. WELSH, RIVERSIDE, IA (US); JOHN A. WEMMIE, IOWA CITY, IA (US); ADAM E. ZIEMANN, IOWA CITY, IA (US)

Correspondence Address: MCKEE, VOORHEES & SEASE, P.L.C., 801 GRAND AVENUE, SUITE 3200, DES MOINES, IA, 50309-2721, US

(73) Assignee: UNIVERSITY OF IOWA RESEARCH FOUNDATION, IOWA CITY, IA (US)

(21) Appl. No.: 12/469,037

(22) Filed: May 20, 2009

Related U.S. Application Data
(60) Provisional application No. 61/055,076, filed on May 21, 2008.

(51) Int. Cl. A61K 33/00; G01N 33/53; A61P 25/08

(52) U.S. Cl. 424/700; 435/7.21

(57) ABSTRACT

This invention provides novel methods and compositions for treating and preventing seizures by administration of ASIC1a receptor activating compounds. A novel method of assaying ASIC1a receptor activating compounds is included in the present invention. According to the invention applicants have demonstrated that seizure duration, intensity, and progression may be modulated by administration of an ASIC1a receptor activator which acts to increase the endogenous activity of ASIC1a receptors to mediate the effects of low pH in the CNS. The inventors have also found that the ASIC1a receptor activator may prevent such seizures altogether.

### CROSS-REFERENCE TO RELATED APPLICATIONS

This application claims priority under 35 U.S.C. § 119 of a provisional application U.S. Ser. No. 61/055,076 filed May 21, 2008, which application is hereby incorporated by reference in its entirety.

### GRANT REFERENCE

This invention was made with government support under Federal Grant No. 1R21NS058309. The United States government has certain rights in the invention.

### FIELD OF THE INVENTION

This invention relates to acid-sensing ion channel 1a (ASIC1a) agonists and other receptor activating means for increasing activity of ASIC, preferably ASIC1a to treat and prevent seizures. In particular, this invention relates to methods of and compositions for treatment and prevention of seizure disorders, including but not limited to, generalized seizure disorders, acute seizures, tonic-clonic seizures, and combinations of the same, by decreasing seizure severity, duration and progression.

### BACKGROUND OF THE INVENTION

The present invention relates to pharmaceutical compositions and methods for treating central nervous system (CNS) disorders, specifically seizures. CNS disorders, including seizures, are attributable to many causes. Disorders can be drug induced, attributed to genetic predisposition, infection or trauma, and are often of unknown etiology. The present invention discloses novel methods and compositions for the treatment and prevention of seizures. The inventors have surprisingly shown that specific ASIC channel activators that modulate ASIC activity, preferably, increasing ASIC activity, are useful in treating and preventing forms of epilepsy.

Epilepsy is one of the most common of the serious neurological disorders. Genetic, congenital, and developmental conditions are mostly associated with it among younger patients; tumors are more likely over age 40; head trauma and CNS infections may occur at any age. The prevalence of epilepsy is approximately 5 to 10 per 1000 people. Up to 50 per 1000 people experience non-febrile seizures at some point in life; whereas epilepsy's lifetime prevalence is relatively high because most patients either stop having seizures or eventually die. Epilepsy's approximate annual incidence rate is 40 to 70 per 100,000 in industrialized countries and 100 to 190 per 100,000 in resource-poor countries.

Epilepsy is not considered to be a single disorder; rather it covers a wide spectrum of problems and disorders characterized by unprovoked, recurring seizures that are disruptive to the normal neurological function of an individual. It is understood that epileptic seizures occur when a group of neurons in the brain become activated simultaneously, emitting sudden and excessive bursts of electrical energy. Such hyperactivity of neurons occurs in various locations throughout the brain, and depending on the location have a wide range of effects on the individual. Seizures can be brief, lasting only a few seconds, or can include minor spasms with or without consciousness. The effects of seizures can also include more severe and significant outcomes such as bodily injury due to major spasms, progressing to status epilepticus which can lead to death.

The various types of seizures can be divided into various subclasses including generalized seizures (further including absence, atonic, tonic-chronic, and myoclonic seizures), partial seizures (further including simple and complex seizures), non-epileptic seizures and status epilepticus. Although there are various categories and subcategories of seizures, therapeutic approaches and accurate diagnoses require the understanding that epilepsy can include a combination and variation of the categories and subtypes. The present invention is directed towards the treatment and prevention of all forms of epilepsy through the application and use of ASIC1a mediated activity in the CNS.

It has been recognized that rapid acidification of extracellular pH in CNS disorders evokes a transient cation current in the central neurons (Groul et al., 1980; Krishtal and Pidoplichko, 1981). Due to the brain's pH being tightly regulated in vivo, the physiological significance of this observation has been unclear to date. It had been hypothesized only that H⁺-gated currents within the brain might be activated during synaptic transmission due to acidifying the extracellular fluid in hippocampal slices (Krishtal et al., 1987). The discovery of acid-sensing ion channels (ASICs), acid-sensing members of the degenerin/epithelial Na+ channel (DEG/ENaC) family, has presented an opportunity to further explore the unknown physiological role of neuronal H⁺-evoked currents.

Overall, very little is known about how the brain limits seizure duration, and there is a scarcity of knowledge about the mechanisms that terminate seizures despite the consequences of a failure to stop seizures. The present invention discovers how the ASIC1a contributes to mediation of the physiological decrease in brain pH during seizures to promote termination and a method for treating and preventing seizures through the use of various ASIC1a receptor activators. The present invention thus teaches the previously unknown effect of ASIC1a mediation in the brain, providing methods and compositions of treatment for seizures and seizure disorders.

Limited research has indicated that seizures produce inhibitory compounds that block continued seizure activity. For example, protons are an inhibitor that accumulates during seizures. Additionally, lactic acid production, CO₂ accumulation, and other factors reduce brain pH from approximately 7.35 to less than 7.0 during a seizure. Acidosis was first implicated in seizure inhibition in 1929 when Lennox found that hypercarbic acidosis eliminated seizure discharges in patients with epilepsy, a finding verified by others. Similarly, the anticonvulsant acetazolamide reduces extracellular pH in the brain.

Prior research concluded that ASIC worked to treat seizures by the opposite effect, by inhibiting the effects of acidosis, rather than effectuating the seizure-terminating effects of acidosis in the CNS as claimed by the present invention. The applicants' own prior research and that of others, lead to the conclusion that the treatment and prevention of seizures was improved by ASIC antagonists, as incorporated by reference in its entirety from Welch et al. 20070087964. For example, the claims directed toward pharmaceutical compositions for treatment and prevention of seizures all required the presence of an ASIC receptor antagonist and a pharmaceutically acceptable carrier. Similarly, method claims directed toward treating or preventing seizures involved administering a therapeutically effective amount of an ASIC antagonist. The applicant's prior invention identified pharmacological agents that block (antagonists) ASIC could inhibit the damaging effects of acidosis and excess glutamate release, occurring during seizures. Such strong evidence of nonobviousness of the present invention shows that the present invention is the result of not only a long-felt and unmet need in the area of seizure and seizure disorder treatment, but also illustrates the failure of others skilled in the art. Moreover, the present invention demonstrates highly unexpected results.

While the science of studying seizures and epileptic effects has advanced significantly, there remains a crucial need for additional neurotransmission research to determine safe and efficient means for treating and preventing seizures, as there are no commercially viable means for the prevention and eradication of the disorder currently available. For the foregoing reasons, there is a need to provide methods of treatment and prevention of seizures.

Accordingly, it is an objective of the invention to provide a method of treatment for seizures using ASIC modulation, such as ASIC receptor activator mediation of seizure-terminating activity of brain acidosis.

A further objective of the invention is a method of decreasing the duration and severity of seizures.

A further objective of the invention includes methods and compositions for minimizing the progression of seizures.

Yet another objective of the invention includes methods and compositions for preventing status epilepticus seizures from initiating in the CNS.

A still further objective of the invention is a method for inhibiting neuron excitability using ASIC agonists.

A still further objective of the invention is a method for raising action potential threshold using ASIC agonists.

A still further objective of the invention is a method for increasing post-ictal depression associated with seizure termination using ASIC agonists.

Another objective of the invention is a method for treating and preventing seizures and seizure disorders by administering new therapeutic agents able to modulate, actuate, over express, or combinations of the same, the ASIC1a channel.

Another objective is to provide and manufacture pharmaceutical compositions for the treatment of seizures using ASIC receptor activators.

### SUMMARY OF THE INVENTION

The present invention identifies the function of acid-gated currents in general and H+-gated DEG/ENaC channels that potentiates the effects of acid-sensing ion channels molecular identity and physiologic function which has remained unknown until now thereby allowing for new methods of treatment of seizures and seizure disorders.

ASIC channels are inhibitory neurons expressed in excitatory pyramidal neurons, likely involved in the depolarization blockade. ASIC channels are located in the hippocampus and many other regions of the brain, making it possible that ASIC activation of inhibitory neurons in other regions may also contribute to seizure termination and provide methods and compositions for seizure disorders and treatments. Specifically, pharmaceutical agents that potentiate ASIC1a's protective activity in brain physiology would result in reduced seizure severity and duration as well as the prevention status epilepticus.

The present invention is directed to compositions and methods for treatment and prevention of seizures and seizure disorders by providing ASIC receptor activators, preferably ASIC1a receptor activators such as agonists. According to the invention, a method for treating seizures comprises administering to a patient in need thereof a therapeutically effective amount of an ASIC receptor activator, preferably an ASIC1a receptor activator and a pharmaceutically acceptable carrier. The pharmaceutically effective amount of an ASIC1a receptor activator can include a direct ASIC1a receptor enhancer (such as, for example, a chemical compound acting as an agonist, introduction of a DNA sequence encoding for ASIC1a or any other ASIC1a enhancer), indirect modulation by interacting with the ASIC pathway, or any other means capable of increasing receptor activity.

According to the invention, a pharmaceutical composition for preventing, treating, or decreasing the duration and severity of seizures comprises a therapeutically effective amount of an ASIC1a receptor activator and a pharmaceutically acceptable carrier. In addition, the present invention also relates to a screening protocol for identifying new therapeutic agents based on their ability to act as an ASIC agonist and/or increase the receptor activity. Finding an ASIC agonist is suggested through protein localization utilizing immunohistochemistry, thereby assaying to provide a treatment for seizures and seizure disorders are presented.

### DEFINITIONS

For purposes of this application the following terms, as used herein, shall have the definitions recited herein. Additionally, all units, prefixes, and symbols may be denoted in their SI accepted form, as well as numeric ranges in the application are inclusive of the numbers defining the range and include each integer within the defined range. The terms defined below are more fully defined by reference to the specification as a whole.

The term “ASIC1a agonist” includes any compound which causes activation of the ASIC1a. This includes both competitive and non-competitive agonists as well as prodrugs which are metabolized to ASIC1a agonists upon administration, as well as analogs of such compounds disclosed by the assays enclosed herein to be active ASIC1a agonists.

The term “ASIC1a receptor activator” includes any compound causing activation or receptor activity, expression, over-expression, or modulation of the ASIC1a receptor, either directly or indirectly. This includes all forms of agonists (i.e., including both competitive and non-competitive agonists), prodrugs which are metabolized to ASIC1a agonists, analogs of such compounds, and transgenic versions of the same.

The term “pharmaceutically acceptable carrier” refers to any carrier, diluent, excipient, wetting agent, buffering agent, suspending agent, lubricating agent, adjuvant, vehicle, delivery system, emulsifier, disintegrant, absorbent, preservative, surfactant, colorant, flavorant, or sweetener, preferably non-toxic, that would be suitable for use in a pharmaceutical composition.

The term “pharmaceutically acceptable equivalent” includes, without limitation, pharmaceutically acceptable salts, hydrates, metabolites, prodrugs and isosteres. Many pharmaceutically acceptable equivalents are expected to have the same or similar in vitro or in vivo activity as the compounds of the invention.

The terms “pharmaceutically effective” or “therapeutically effective” shall mean an amount of each active component of the pharmaceutical composition (i.e., ASIC1a receptor activator) or method that is sufficient to show a meaningful patient benefit (i.e., treatment, prevention, amelioration, or a decrease in the frequency of the condition or symptom being treated), as determined by the methods and protocols disclosed herein. When applied to an individual active ingredient, administered alone, the term refers to that ingredient alone. When applied to a combination, the term refers to combined amounts of the active ingredients that result in the therapeutic effect, whether administered in combination, serially or simultaneously.

The terms “treat” or “treating”, unless otherwise defined in conjunction with specific diseases or disorders, refers to: (i) inhibiting the disease, disorder or condition, i.e., arresting its development; (ii) relieving the disease, disorder or condition, i.e., causing regression, decrease in severity and/or progression; (iii) terminating an episode or event caused by the disease, disorder and/or condition; and/or (iv) preventing a disease, disorder or condition from occurring in an animal or human that may be predisposed to the disease, disorder and/or condition but has not yet been diagnosed as having it.

### BRIEF DESCRIPTION OF THE DRAWINGS

FIG. 1a demonstrates seizure response scored using the Racine seizure scale over time in ASIC1a+/+ and ASIC1a−/− mice following 20 mg/kg kainate injected into the peritoneum (IP). Seizures became more severe in ASIC1a−/− mice, showing that ASIC1a reduces seizure severity.

FIG. 1b demonstrates the maximum Racine score during the 60 minute trial.

FIG. 1c shows the incidence of generalized tonic-clonic seizures (GTCS) in ASIC1a+/+ and ASIC1a−/− mice following 50 mg/kg IP pentylenetetrazole (PTZ). The incidence of GTCS was significantly greater in ASIC1a−/− mice, showing that ASIC1a reduces incidence of GTCS and overall seizure severity.

FIG. 1d shows PcTx1 increased the incidence of continuous GTCS following kainate injection.

FIG. 1e shows ASIC1a over-expression reduces seizure severity following kainate injection.

FIG. 1f further shows ASIC1a over-expression reduces seizure severity following kainate injection.

FIG. 1g shows the incidence of GTCS in WT and Tg+ mice following 65 mg/kg IP PTZ.

FIG. 1h shows ASIC1a disruption does not reduce the amount of electrical current required for initial seizure threshold.

FIG. 2a shows representative electroencephalography (EEG) tracings and quantification of time from IP PTZ until first seizure spikes in ASIC1a+/+ and ASIC1a−/− mice.

FIG. 2b shows representative EEG tracings and total number of seizure spikes per five minute interval in surviving mice, demonstrating that ASIC1a−/− mice had prolonged seizure activity as time elapsed.

FIG. 2c demonstrates that the incidence of GTCS increases and the percent survival decreases with increasing ASIC1a disruption.

FIG. 2d shows the survival over time (+/+, n=6; −/−, n=7; Mantel-Cox Log Rank, p=0.025).

FIG. 3a demonstrates representative EEG tracings from an ASIC1a+/+ and ASIC1a−/− mouse after IP PTZ.

FIG. 3b further shows an expanded view of the EEG tracings prior to PTZ injection, initial spike wave activity, seizures, immediately following seizures, post-ictal depression and seizure activity.

FIG. 3c shows the quantification of post-ictal depression.

FIG. 4a shows there is no epileptiform activity in ASIC1a+/+ and ASIC1a−/− neuron slices prior to induction of seizures.

FIG. 4b shows representative CA3 extracellular recording from ASIC1a+/+ and ASIC1a−/− mice before, during, and after the pH in hippocampal slices is decreased, demonstrating ASIC1a mediation of the antiepileptic effects of acid.

FIG. 4c demonstrates the latency to seizure onset recorded in response to hypomagnesemia.

FIG. 4d demonstrates the total number of seizure spikes at varying pH levels, showing that only ASIC1a+/+ mice were significantly affected by pH change.

FIG. 5a depicts acid-evoked current in an ASIC1a+/+ and ASIC1a−/− neuron in response to low pH.

FIG. 5b shows reductions in pH evoke ASIC currents in interneurons with larger H+-gated current densities than pyramidal neurons.

FIG. 5c demonstrates the reduction of extracellular pH stimulates action potential firing in inhibitory neurons.

FIG. 6a shows Kaplan-Meier survival analysis of ASIC1a+/+ and ASIC1a−/− mice in response to PTZ while breathing compressed air.

FIG. 6b shows a parallel experiment to FIG. 6a, where 10% CO₂ was administered at the onset of GTCS. The likelihood of survival was significantly greater in the ASIC1a+/+ mice.

FIG. 6c shows that generalized seizures caused brain pH to decrease.

FIG. 6d also shows that generalized seizures caused brain pH to decrease.

### DETAILED DESCRIPTION OF THE PREFERRED EMBODIMENT

According to the invention, seizures reduce brain pH and acidosis inhibits seizures, indicating that decreasing the brain's pH halts epileptic activity. Specifically, the present invention is directed to the ASIC1a mediation of seizure-terminating activity of brain acidosis. For example, disrupting mouse ASIC1a increased the severity of chemoconvulsant-induced seizures, whereas over-expressing ASIC1a decreased seizure severity. Although ASIC1a expression does not appear to affect the onset of seizures, it shortens the duration and prevents progression of such seizures. Moreover, inhibitory effects of acidosis on epileptiform activity in brain slices and action potential threshold are ASIC1a-dependent. In addition, CO₂ inhalation requires ASIC1a to interrupt lethal tonic-clonic seizures.

Acidosis inhibits seizures through multiple mechanisms. For example, extracellular acidosis inhibits N-methyl-D-aspartic acid (NMDA) receptors, and NMDA receptor antagonists attenuate acid's effect on epileptiform activity in brain slices. A reduced extracellular pH also inhibits voltage-gated Na+ and Ca2+ channels and modulates gamma-amino butyric acid (GABA_{A}) receptors. Additionally, extracellular acidosis increases the concentration of extracellular adenosine, activating adenosine (A1) receptors and ATP (P2X and P2Y) receptors to reduce seizure-like activity in brain slices. The ability of extracellular acidosis to activate ASIC1a shows that the proteins also mediate the effects of pH on seizures.

ASICs are proton-gated members of the DEG/ENaC family of Na.sup.+ permeable channels, which includes the FMRFamide-gated channel (FaNaCh). The brain expresses at least three ASICs: ASIC1a, -2a, and -2b. These form homo- and heteromultimeric channels widely expressed in the CNS. They are activated by a drop of pH below 6.8 and desensitize rapidly which has raised the question of their functional role (Akaike et al., 1994). The current invention utilizes the finding that ASIC, specifically ASIC1a, decreases seizure duration, severity and progression through its mediation of the physiological decrease in pH resulting from seizures. ASIC1a homomeric channels are activated by protons and conduct Na⁺ and Ca²⁺ with an EC50 of approximately 6.8. In CNS neurons, ASIC1a is required to generate a current response to pH values between approximately 7.2 and 5.0, illustrating ASIC1a's critical role in mediating the brain's response to acidosis. Consistent with its role in modulating neuron excitability, the present invention shows that ASIC1a can activate or inhibit neuron firing, contrary to an earlier study showing that inhibitory interneurons had larger H+-gated currents than excitatory neurons, suggesting ASICs would dampen excitability (Bolshakov et al., Neuroscience, 2002).

The identification of ASIC1a as a channel capable of terminating and preventing progression of seizures opens new opportunities to investigate the poorly understood molecular mechanisms responsible for terminating seizures. Based on these findings, therapeutic agents capable of binding, activating, or causing expression of ASIC1a can terminate, treat and prevent seizures and seizure disorders. By identifying a key ion channel, the present invention identifies a molecular mechanism for how the brain stops seizures. Additionally, the present invention provides for new therapeutic strategies for treating various seizure disorders.

Acid-activated cation currents have been detected in central and peripheral neurons for more than 20 years (Gruol et al., 1980; Krishtal and Pidoplichko, 1981). In the CNS, they have been observed in the hippocampus (Vyldicky et al., 1990), cerebellum (Escoubas et al., 2000), cortex (Varming, 1999), superior colliculus (Grantyn and Lux, 1988), hypothalamus (Ueno et al., 1992), and spinal cord (Gruol et al., 1980). Currents evoked by a fall in extracellular pH vary in pH sensitivity, with half maximal stimulation ranging from pH 6.8 to 5.6 (Varming, 1999). Despite the wide spread distribution of H.sup.+-gated currents in the brain, neither their molecular identity nor their physiologic functions are known.

Although many central neurons possess large acid-activated currents, their molecular identity and physiologic function have remained unknown. Previous to the discovery of ASIC receptors, the NMDA receptor has been implicated during development in specifying neuronal architecture and synaptic connectivity and may be involved in experience dependent synaptic modifications.

Researchers have identified a family of cation channels that are gated by reductions in pH. These proteins, called ASICs, are related to amiloride-sensitive epithelial sodium channels (ENaCs) and the degenerin/mec family of ion channels from Caenorhabditis elegans (Waldmann et al., 1997). The acid-sensing DEG/ENaC respond to protons and generate a voltage-insensitive cation current when the extracellular solution is acidified. Prior inventions have found the ASIC in the hippocampus, enriched in synaptosomes, and localized at dendritic synapses in hippocampal neurons (Welsch et al., 20070087964).

There has been speculation about the physiologic and pathophysiologic function of acid-gated currents in central neurons. It has been hypothesized that interstitial acidosis associated with seizures and ischemia could trigger their activity, thereby exacerbating the pathological consequences of these conditions (Biagini et al., 2001; Ueno et al., 1992; Varming, 1999; Waldmann et al., 1997b). Although macroscopic changes in extracellular pH in the brain are tightly controlled by homeostatic mechanisms (Chesler and Kaila, 1992; Kaila and Ransom, 1998) it is possible that pH fluctuations in specific micro-domains such as the synapse may be significant (Waldmann et al., 1997b). For example, the acid pH of synaptic vesicles has been suggested to transiently influence local extracellular pH upon vesicle release (Krishtal et al., 1987; Waldmann et al., 1997b). Consistent with this idea, transient acidification of extracellular pH has been recorded with synaptic transmission in cultured hippocampal neurons (Miesenbock et al., 1998; Ozkan and Ueda, 1998; Sankaranarayanan et al., 2000) and in hippocampal slices (Krishtal et al., 1987). Thus it has been suggested that acid-evoked currents may play a role in the physiology of synaptic transmission (Krishtal et al., 1987; Waldmann et al., 1997b).

DEG/ENaC channels activated by a reduction in extracellular pH play diverse physiologic roles. The ability of these channels to respond to different stimuli and to serve different cellular functions may depend on their multimeric subunit composition, their location, associated proteins, and the cellular context. However, in the CNS, the function of acid-gated currents in general and H.sup.+-gated DEG/ENaC channels in particular has remained unknown. The present studies provide insight into the function of these channels, specifically the ASIC1a, in the CNS.

Applicants injected ASIC1a+/+WT and ASIC1a−/− mice with various chemoconvulsants to assess seizure severity, duration, progression, post-ictal depression, and action potential threshold in order to demonstrate the seizure-terminating effects of the present invention (Examples 1-10).

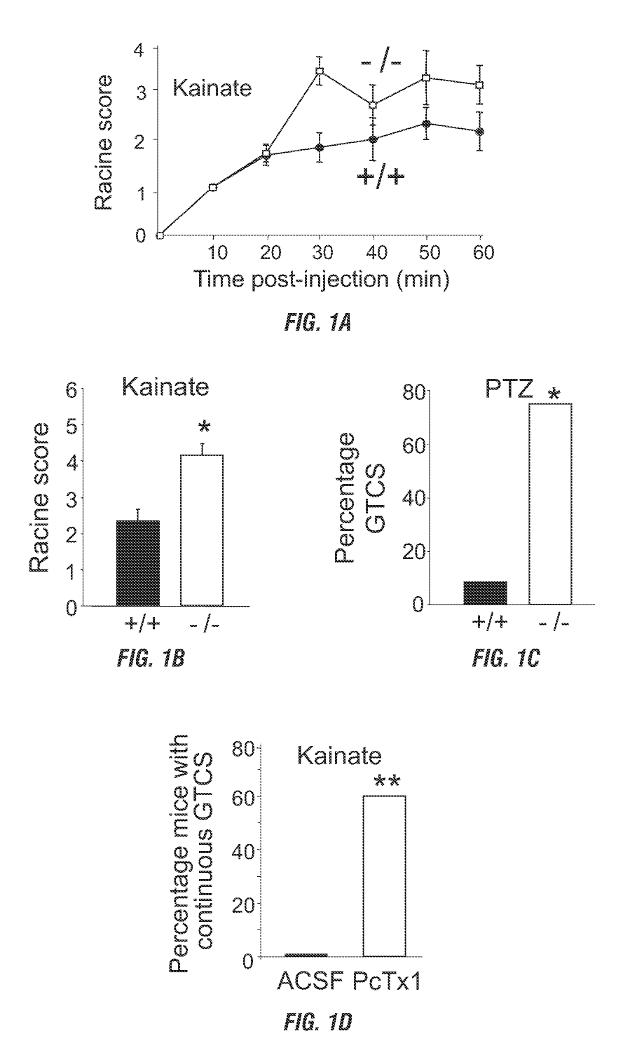

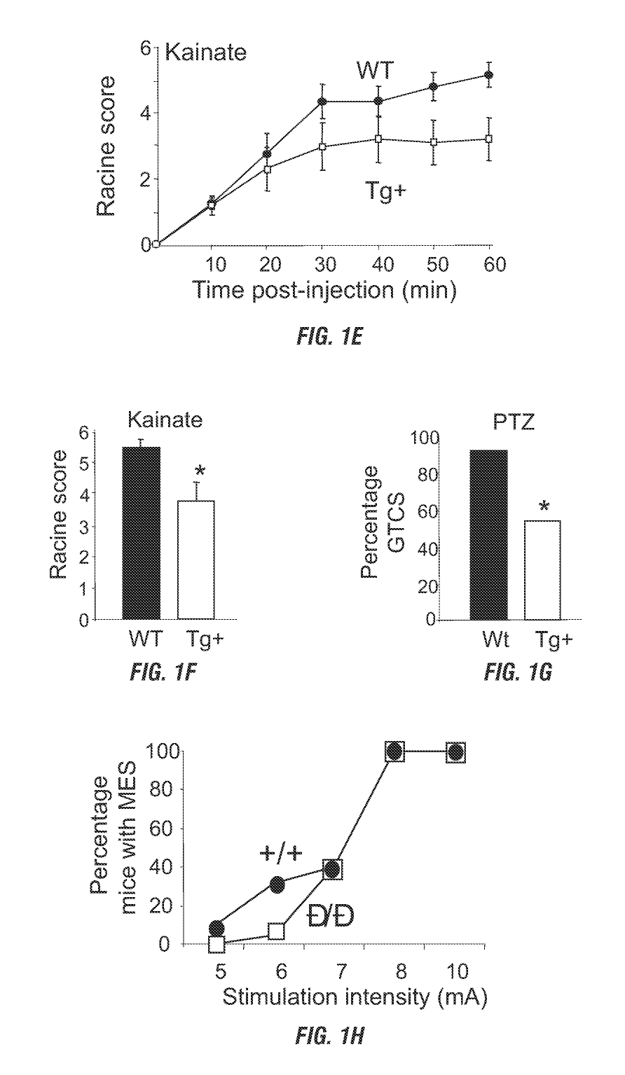

According to the invention, applicants have discovered that ASIC1a disruption increases seizure severity, whereas ASIC1a activation or an ASIC1a agonist reduces seizure severity. Test subjects with both genotypes show similar seizure responses upon initial injection of a chemoconvulsant; however with time, ASIC1a-null mice develop significantly more severe seizures (FIG. 1), demonstrating that ASIC1a reduces seizure severity. Notably, the use of two different chemoconvulsants (kainate and PTZ) results in decreased seizure severity for mice expressing ASIC1a channels.

The present invention also demonstrates that ASIC1a over-expression reduces seizure severity. Applicants found that ASIC1a disruption enhanced seizure severity and concomitantly, over-expressing the channel has the opposite effect. ASIC1a over-expression also reduced the incidence of GTCS in addition to generally reducing seizure severity (FIG. 1g).

According to the invention an ASIC enhancer may also be used to shorten seizure duration in a patient already experiencing seizure activity. PTZ-evoked seizures, using EEG to examine epileptiform discharges while simultaneously monitoring seizures behaviorally, were assessed to determine how ASIC1a reduces seizure severity. Disrupting ASIC1a prolonged EEG spike activity and increase the likelihood that seizures progress to GTCS or death. As such the invention includes methods and compositions for shortening seizure duration by administering to a patient a pharmaceutically effective amount of an ASIC receptor activator, such as an ASIC1a agonist.

The present invention still further demonstrates modulation of ASIC1a may be used to termination of seizure-like activity. The reduced pH occurring during seizures terminates the seizure through ASIC1a. Seizures in WT mice were followed by a suppression of spike discharges, commonly referred to as post-ictal depression. The post-ictal depression reverts to seizure activity in the absence of ASIC1a channels. The loss of an EEG pattern associated with seizure termination is consistent with the prolonged seizure activity and the increased severity resulting from the disruption of ASIC1a channels necessary to reduce and terminate seizure activity.

The present invention additionally demonstrates that acid-induced elevation of action potential threshold, a critical factor in the termination and decrease in severity of seizures, is ASIC1a-dependent. Acidosis interrupts action potential firing and most neuron excitability regulation, necessary for seizure activity. Moreover, action potential generation occurs in the dendrites and cell body where ASIC1a channels are localized. The present invention demonstrates that ASIC1a activation decreases excitability by inhibiting action potential generation via membrane potential depolarization to cease action potential firing, responsible for decreasing seizure activity in order to promote the termination and decrease in severity and progression of seizures.

The effects of ASIC1a and low pH on action potential threshold were also tested to demonstrate the impact on termination and decrease in severity of seizures. As pH decreased, action potential frequency, amplitude, number, and duration were similar between ASIC1a+/+ and ASIC1a−/− neurons. However, neurons from the two genotypes exhibited different thresholds for action potential generation, with the firing threshold of ASIC1a+/+neurons elevated, resulting in a decrease in CNS activity responsible for seizures. Therefore, the present invention additionally demonstrates that ASIC1a channels are required for acidosis to raise action potential threshold and thereby reduce excitability and seizure activity.

The effects of ASIC1a were not shown to significantly affect the seizure threshold for affecting seizure initiation or onset. ASIC1a disruption fails to affect seizure threshold, latency to seizure onset, or initial seizure severity. The maximal electroconvulsive seizure threshold test was used to as a method for threshold analysis, in ASIC1a+/+ and ASIC1a−/− mice. Electroshock was delivered (0.2 seconds, 60 Hz, maximal voltage 500 V) using the Rodent Shocker-type 221 (Harvard Apparatus, Holliston, Mass.) with ear electrodes moistened with saline. The occurrence of generalized seizures with sustained hind limb extension was assessed. ASIC1a disruption did not reduce the amount of electrical current necessary to evoke a stereotypic seizure response (FIG. 1h), indicating that ASIC1a does not play a prominent role in determining initial seizure threshold.

Another aspect of the present invention is that carbon dioxide inhalation utilized to interrupt or stop seizures requires ASIC1a mediation. Therefore, yet another aspect of the invention is potentiating CO₂ inhibition of seizures by administering CO₂ in the presence of ASIC1a agonist. Inhaling CO₂ inhibits seizures in humans, as studies have demonstrated that CO₂ reduces cortical pH within seconds of inhalation, and that breathing CO₂ increases brain acidosis during a PTZ-evoked seizure. The inventors discovered that inducing hypercarbic acidosis requires ASIC1a for the antiepileptic effects of acidosis.

Extracellular acidosis activates ASIC1a which reduces seizure activity. Three different chemoconvulsants (kainate, PTZ, and a reduced Mg²⁺ concentration (hypomagnesemia)) to trigger epileptiform activity. In addition to the acidosis generated by seizures, the extracellular pH in vitro was directly lowered and CO₂ was administered in vivo to generate brain acidosis; both ending seizure activity in an ASIC1a-dependent manner. Additionally, disrupting ASIC1a increased seizure severity, whereas over-expressing ASIC1a had the opposite effect, verifying that ASIC1a forms part of a feedback inhibition system that limits seizure severity. These findings provide for the methods and compositions of the present invention to treat seizures and seizure disorders.

The present invention also includes methods for treating seizures using a therapeutically effective amount of an ASIC1a receptor activator. For example, the term receptor activator includes any compound which causes the activation or increased activity of the ASIC1a receptor in the CNS. This includes all forms of agonists, pro-drugs, receptor modulators, and expression enhancers. Additionally included are all forms of the same that are metabolized into receptor agonist modulators or enhancers upon administration, as well as analogs and pharmaceutically acceptable equivalents of the same compounds.

The invention includes a method and pharmaceutical compositions which includes an ASIC1a receptor activator and a carrier which may be administered to a patient in need thereof. In addition to administration with conventional carriers, active ingredients may be administered by a variety of specialized delivery drug techniques which are known to those of skill in the art. The following examples are given for illustrative purposes only and are in no way intended to limit the invention.

ASIC1a receptor activators may be identified by means known to those of skill in the art, compositions which bind to the channels can be identified or designed (synthesized) based on the disclosed knowledge of potentiation of the channels and determination of the three-dimensional structure of the channels, as incorporated by reference in its entirety from Welch et al. 20070087964. These compositions act as agonists, expression enhancers, or modulators affecting a decrease in seizure progression, severity, duration and all effects described in the present invention.

The pharmaceutical preparations of the present invention are manufactured in a manner which is itself well known in the art. For example, the pharmaceutical preparations may be made by means of conventional mixing, granulating, dragee-making, dissolving, and lyophilizing processes. The processes to be used will depend ultimately on the physical properties of the active ingredient used.

In addition to the active compounds (i.e., ASIC1a receptor activators), the pharmaceutical compositions of this invention may contain suitable excipients and auxiliaries which facilitate processing of the active compounds into preparations which can be used pharmaceutically. Oral dosage forms encompass tablets, dragees, capsules and other pharmaceutically acceptable carriers. Preparations which can be administered rectally include suppositories. Other dosage forms include suitable solutions for administration parenterally (both intravenously and intramuscularly) or orally, and compositions which can be administered buccally or sublingually.

Suitable excipients are, in particular, fillers such as sugars for example, lactose or sucrose mannitol or sorbitol, cellulose preparations and/or calcium phosphates, for example, tricalcium phosphate or calcium hydrogen phosphate, as well as binders such as starch, paste, using, for example, maize starch, wheat starch, rice starch, potato starch, gelatin, gum tragacanth, methyl cellulose, hydroxypropylmethylcellulose, sodium carboxymethylcellulose, and/or polyvinyl pyrrolidone. If desired, disintegrating agents may be added, such as the above-mentioned starches as well as carboxymethyl starch, cross-linked polyvinyl pyrrolidone, agar, or alginic acid or a salt thereof, such as sodium alginate. Auxiliaries are flow-regulating agents and lubricants, for example, such as silica, talc, stearic acid or salts thereof, such as magnesium stearate or calcium stearate and/or polyethylene glycol. Dragee cores may be provided with suitable coatings which, if desired, may be resistant to gastric juices.

For this purpose concentrated sugar solutions may be used, which may optionally contain gum arabic, talc, polyvinylpyrrolidone, polyethylene glycol and/or titanium dioxide, lacquer solutions and suitable organic solvents or solvent mixtures. In order to produce coatings resistant to gastric juices, solutions of suitable cellulose preparations such as acetylcellulose phthalate or hydroxypropylmethylcellulose phthalate, dyestuffs and pigments may be added to the tablet of dragee coatings, for example, for identification or in order to characterize different combination of compound doses.

Other pharmaceutical preparations which can be used orally include push-fit capsules made of gelatin, as well as soft, sealed capsules made of gelatin and a plasticizer such as glycerol or sorbitol. The push-fit capsules can contain the active compounds in the form of granules which may be mixed with fillers such as lactose, binders such as starches, and/or lubricants such as talc or magnesium stearate and, optionally, stabilizers. In soft capsules, the active compounds are preferably dissolved or suspended in suitable liquids, such as fatty oils, liquid paraffin, or liquid polyethylene glycols. In addition stabilizers may be added.

Pharmaceutical preparations which can be used rectally include, for example, suppositories, which consist of a combination of the active compounds with the suppository base. Suitable suppository bases are, for example, natural or synthetic triglycerides, paraffinhydrocarbons, polyethylene glycols, or higher alkanols. In addition, it is also possible to use gelatin rectal capsules which consist of a combination of the active compounds with a base. Possible base material includes for example liquid triglycerides, polyethylene glycols, or paraffin hydrocarbons.

Suitable formulations for parenteral administration include aqueous solutions of active compounds in water-soluble or water-dispersible form. In addition, suspensions of the active compounds as appropriate oily injection suspensions may be administered. Suitable lipophilic solvents or vehicles include fatty oils for example, sesame oil, or synthetic fatty acid esters, for example, ethyl oleate or triglycerides. Aqueous injection suspensions may contain substances which increase the viscosity of the suspension, include for example, sodium carboxymethyl cellulose, sorbitol and/or dextran, optionally the suspension may also contain stabilizers.

Suitable formulations for parenteral administration include aqueous solutions of active compounds in water-soluble or water-dispersible form. In addition, suspensions of the active compounds as appropriate oily injection suspensions may be administered, for example intramuscularly. Suitable lipophilic solvents or vehicles include fatty oils for example, sesame oil, or synthetic fatty acid esters, for example, ethyl oleate or triglycerides. Aqueous injection suspensions may contain substances which increase the viscosity of the suspension, include for example, sodium carboxymethyl cellulose, sorbitol and/or dextran, optionally the suspension may also contain stabilizers. In addition to administration with conventional carriers, active ingredients may be administered by a variety of specialized drug delivery techniques which are known to those of skill in the art.

The present invention also provides a method for screening new therapeutic agents for the treatment of seizures and seizure disorders by assaying for the agents' ability to act as an agonist or by any mechanism increase the activity of the ASIC family. The nucleotide sequence encoding for ASIC and more specifically ASIC1a are known, as identified with the REFSEQ: accession NM_{—}007384.2.

The assay comprises administering the composition to be screened to cells expressing acid-gated channels and then determining whether the composition has modulates the acid-sensing channels of the DEG/ENaC family. The determination can be performed by analyzing whether a current is generated in cells containing these channels in the presence of the composition and the acid. This current can be compared to that sustained by the FMRFamide and FMRFamide-related peptides.

In addition to the ASIC channels, it is expected that FMRFamide or FMRFamide related peptides will potentiate acid-evoked activity of other members of the DEG/ENaC cation channel family. The determination of enhancement or inhibition can be done via electrophysical analysis. Cell current can be measured. Alternatively, any indicator assay which detects opening and/or closing of the acid-sensing ion channels can be used such as voltage-sensitive dyes or ion-sensitive dyes. An assay which caused cell death in the presence of the peptide, or agonist, would be the most definitive assay for indicating potentiation of the channels. Assays that measure binding of FMRFamide and related peptides to the channels can identify binding of agonists and modulators of binding. One of ordinary skill in the art would be able to determine or develop assays which would be effective in finding compositions that affect the ASICs.

A composition which activates or inactivates the transient or sustained current present when acid or a related peptide activate the acid-sensing ion channels should be useful as a pharmacological agent. The screening can be used to determine the level of composition necessary by varying the level of composition administered. The composition can be administered before or after addition of the acid or a related peptide to determine whether the composition can be used as a treatment for seizures or seizure disorders. One of ordinary skill in the art would be able to determine other variations on the assay(s).

Unless defined otherwise, all technical and scientific terms used herein have the same meaning as commonly understood by one of ordinary skill in the art to which this invention belongs. Unless mentioned otherwise, the techniques employed or contemplated herein are standard methodologies well known to one of ordinary skill in the art. The materials, processes and examples described in the description of the invention are illustrative only and not intended to be limiting to the scope of the invention in any manner. Modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims.

### EXAMPLES

The present invention is further defined in the following examples which are not intended to limit the invention in any way and are provided only for purposes of illustration. All references cited herein are hereby incorporated in their entirety by reference.

Age and gender-matched WT and ASIC1a−/− mice were used on a congenic C57/B16 background as well as ASIC1aTg+ mice generated previously. These mice had normal brain morphology and either lack or over-express ASIC1a throughout the CNS. Care of the mice met the National Institutes of Health standards and procedures were approved by the University of Iowa Animal Care and Use Committee.

EEG recordings and analysis began by stereotactically implanting two, 3.2 mm stainless steel screws (Stoelting, Wood Dale Ill.) under ketamine/xylazine anesthesia above the left frontal lobe and cerebellum; these electrodes served as an epidural recording electrode and reference/ground electrode respectively (frontal: anteroposterior+1.5 mm, lateral−1.5 mm; cerebellum reference: anteroposterior−6.0 mm). Mice recovered from surgery for at least 1 week, and EEG activity was recorded by tethered connecting leads that allowed the mice to move freely in a sound attenuated recording chamber.

The Racine seizure scale scored seizure severity in ASIC1a+/+ (n=6) and ASIC1a−/− mice (n=7) in response to kainate (ages 13-22 weeks): (0) No response, (1) staring/reduced locomotion, (2) activation of extensors/rigidity, (3) repetitive head and limb movements, (4) sustained rearing with clonus, (5) loss of posture, (6) status epilepticus/death.

Statistical values were expressed as mean±s.e.m. Where indicated, analyses of significance were performed using the unpaired T-test or ANOVA with repeated measures to compare two groups at multiple time points or pH values. For ANOVA, current density data were transformed to log 10 values. The Mann-Whitney U-test (Wilcoxon rank sum) was used to compare two groups of ordinal variables. The Fisher's exact test was used to compare two groups of two categorical variables. Kaplan-Meier analysis and Mantel-Cox log rank were used to assess survival. Probit analysis with 95% confidence intervals were used to calculate the CD50 in threshold experiments. P-values less than 0.05 were considered statistically significant (Microsoft Excel, SPSS).

### Example 1

The convulsants kainate or PTZ solutions were injected via IP following suspension in phosphate buffered saline (Gibco, Carlsbad Calif.) and titration to pH 7.4 with 0.1 M NaOH. Mice were injected with 20 mg/kg kainate and scored for 1 hour by a trained observer blinded to genotype. The highest score per ten-minute interval and the maximum score during the entire trial were assessed. Additionally studies scored the incidence of GTCS which were identified by 4-limb explosive clonus followed by tonic hind limb extension.

Dose, genotype, and age were as follows: (1) PTZ 50 mg/kg, ASIC1a+/+ (n=12) vs. ASIC1a−/− (n=8), ages 18-22 weeks; (2) PTZ 65 mg/kg, Tg+(n=13) vs. WT litter-mates (n=13), ages 25-41 weeks; (3) Kainate 30 mg/kg, Tg+(n=10) vs. WT litter-mates (n=13), ages 31-36 weeks. Different kainate and PTZ doses were used to decrease the overall number of animals required and avoid ceiling and floor effects.

EEG was recorded at baseline and in response to a single IP injection of PTZ (50 mg/kg) in gender and age-matched (18-22 week) ASIC1a+/+ and ASIC1a−/− mice. During the 30 minutes following injection, tonic-clonic and lethal seizures were identified behaviorally and electrographically by simultaneous video and EEG monitoring. EEG was captured using a TDT MEDUSA preamplifier and base-station and recorded at a sampling rate of 508.6 Hz with TDT OpenX software with high and low pass filters at 2 Hz and 70 Hz, respectively. EEG recordings were analyzed using Origin 7.5 software by an experimenter blinded to genotype. Latency to seizure onset was defined as the time from injection to first seizure spike. Seizure spikes were detected using the peak analysis function of Origin v7.5. Both major seizure events and sharply delimited seizure spikes exceeding twice the baseline amplitude were scored.

### Example 2

Horizontal hippocampal slices (400 μm) were prepared from 14 to 24-day-old ASIC1a+/+ and ASIC1a−/− mice similar to methods described previously. Prior to the sectioning, mice were transcardially perfused with a high Mg²⁺/low Ca²⁺ solution chilled to 4° C. (in mM): 4.9 MgSO4, 0.5 CaCl2, 126 NaCl, 5 KCl, 1.25 NaH2PO4, 27.7 NaHCO3, 10 Dextrose, 1.1 MgCl2, pH 7.35 bubbled with 95% O2/5% CO₂. After sectioning, slices were incubated in artificial cerebral spinal fluid (ACSF) for at least 1 hour prior to testing: 126 NaCl, 5 KCl, 1.8 MgSO4, 1.25 NaH2PO4, 27.7 NaHCO3, 10 Dextrose, and 1.6 CaCl2.

Standard extracellular field potential recording techniques were performed in a submerged chamber perfused with ACSF (flow-rate 4 ml/min, 33° C.±0.5° C.). Field-potentials were recorded in the proximal CA3 hippocampal field with ACSF-filled glass pipettes (<5 MΩ). To evoke seizure activity, normal ACSF was replaced with ACSF minus MgSO4. Latency to onset of epileptiform activity was defined as the time elapsed between switching to nominal Mg²⁺ ACSF until the first epileptiform spike. After scoring the latency to epileptiform activity and recording 5 minutes of seizure activity, pH was reduced to 6.8 by lowering NaHCO3 concentration to 11.4 mM, and increasing sodium gluconate to 16.3 mM to maintain osmolarity.

The effects of low pH were recorded for 5 minutes and pH was then switched back to 7.35. Slices that failed to develop ictal discharges were excluded (40% of ASIC1a+/+ slices, n=15, and 38% of ASIC1a−/− slices, n=13). Epileptiform activity was measured using the threshold function in Clampfit v9.2 to quantify the total number of seizure spikes during three 4.5-minute time windows occurring immediately before, during, and after dropping pH to 6.8. The 30 seconds required to change the bath solution to pH 6.8 were excluded from the analysis. A threshold was chosen for each slice that would detect seizure spikes, and not single-unit activity. The average thresholds were similar between genotypes (ASIC1a+/+=0.355±0.032 mV; ASIC1a−/−=0.305±0.04 mV). When slices were challenged repeatedly with low pH, the trials were pooled to calculate a mean number of discharges for each condition.

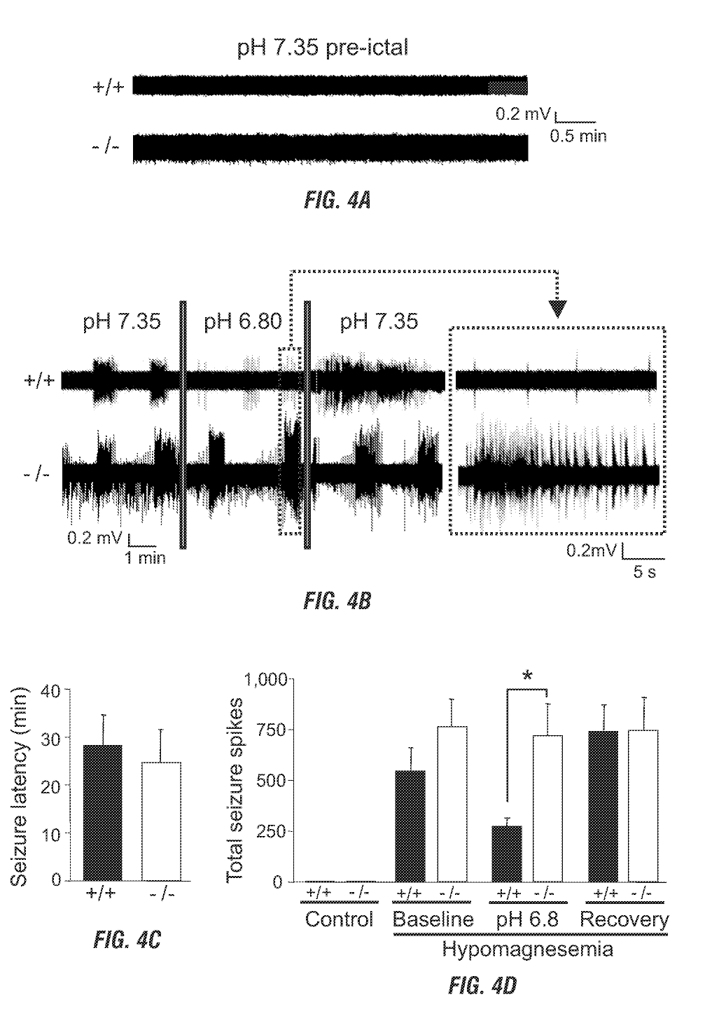

A hippocampal slice model in which hypomagnesemia induces epileptiform activity was utilized. In this model, low pH inhibits seizure-like activity in the hippocampus, and ASIC1a is expressed in hippocampal neurons. Representative CA3 extracellular recording from ASIC1a+/+ and ASIC1a−/− mice before, during, and after pH 6.8 application were recorded (FIG. 4b). A seizure-like discharge during pH 6.8 is expanded in the inset. At pH 7.35, WT and ASIC1a-null slices showed similar latency to the onset of epileptiform activity (FIG. 4b, FIG. 4c) and had an equivalent number of epileptiform spikes (FIG. 4b, FIG. 4d). However, when the pH was reduced to 6.8, seizure activity decreased in WT slices (FIG. 4b, FIG. 4d), not in slices from ASIC1a−/− mice, showing that ASIC1a expression is required for the antiepileptic effects of low pH. Before seizures were induced, no epileptiform activity was observed in slices of either genotype (FIG. 4a).

The latency to seizure onset was recorded in CA3 in response to hypomagnesemia (0 Mg²⁺) (+/+, n=7; −/− n=5; df (10), t=0.366, p=0.722) (FIG. 4c). The total number of seizure spikes over 4.5 minutes at pH 7.35 (baseline), pH 6.8, and after return to pH 7.35 (recovery) (+/+, n=9; −/− n=8) are depicted in FIG. 4d. In ASIC1a+/+ mice an ANOVA revealed a significant effect of pH (df (2), F=7.124, p=0.006), however this was not the case in the ASIC1a−/− mice (df (2), F=0.104, p=0.902). A within-subjects comparison revealed a significant pH×genotype interaction (df (2), F=3.78, p=0.034). At pH 6.8, the spike number was significantly greater in the ASIC1a−/− mice (unpaired T-test: df (15), t 24=−2.88, *p=0.006), whereas at baseline and during recovery, the ASIC1a+/+ and ASIC1a−/− mice did not significantly differ (unpaired T-test: p=0.238 and 0.581 respectively).

### Example 3

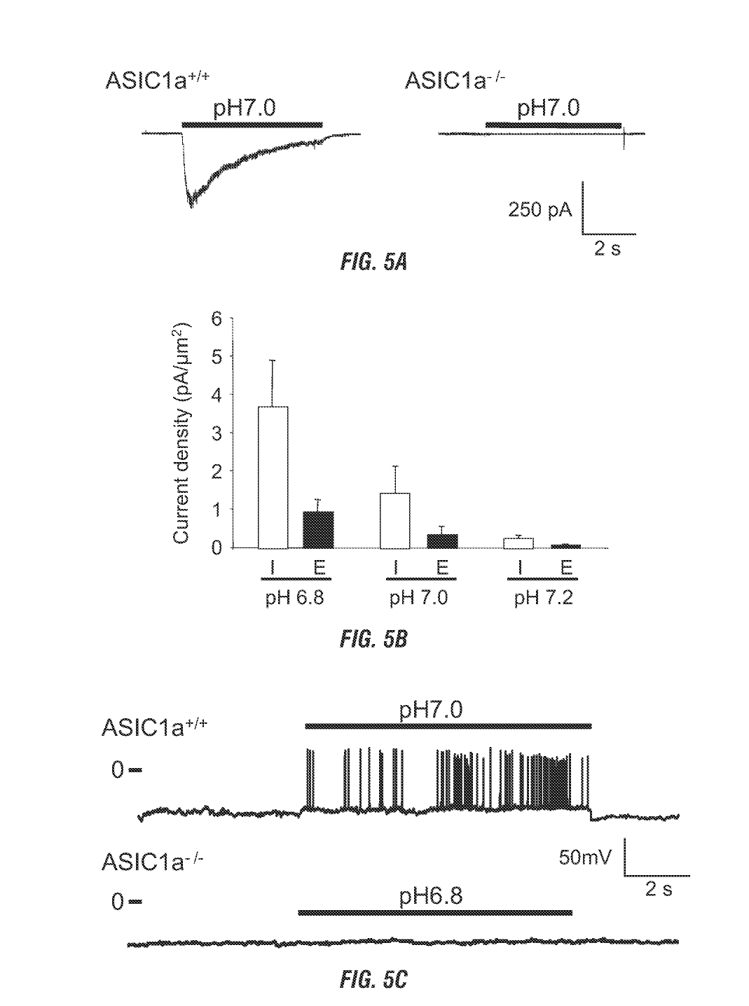

The activation of inhibitory interneurons, cell populations with a critical role in limiting epileptiform activity, was tested as a mechanism by which acidosis-induced ASIC currents could inhibit epileptiform discharges. The effects of physiologically relevant reductions in pH on acutely dissociated hippocampal interneurons, as well as excitatory pyramidal neurons. Neurons were identified based on their location (lacunosum moleculare vs. CA1), size, morphology, and firing pattern, resulting in reduced extracellular pH activating inward current in wild-type, but not ASIC1a−/− interneurons (FIG. 5a), consistent with prior studies showing ASIC1a disruption eliminates currents evoked by pH reductions to as low as 5.0.

Reducing the pH from 7.4 to 7.2, 7.0 and 6.8 also evoked ASIC currents (FIG. 5b). The pH values are those ranges reported in seizures and in the same range measured by Applicants. The interneurons also had larger H+-gated current densities than pyramidal neurons (FIG. 5b), similar to inhibitory interneurons in rats reported to possess larger acid-evoked currents than excitatory neurons.

The reduced extracellular pH also stimulated action potential firing in inhibitory neurons. pH 6.8 induced firing in 78% of inhibitory neurons (n=9), and pH 7.0 induced firing in 80% of the interneurons (n=5) (FIG. 5c), indicating that ASIC1a−/− animals lack a source of inhibitory tone during central acidosis and therefore fail to inhibit seizure activity.

### Example 4

Acutely dissociated neurons were isolated from age 8-12 day old ASIC1a−/− and ASIC1a+/+ mice. Mice were anesthetized (isoflurane), decapitated, and 500 uM coronal sections were cut with a vibratome in ice-cold PIPES buffered saline (115 mL NaCl, 5 mL KCl, 20 mL PIPES, 1 mL CaCl2, 4 mM MgCl2 D-glucose 25, pH 7.0 with NaOH) in the presence of 100% O2. CA1 and the lacunosum moleculare layer (LM) of the hippocampus were removed by microdissection and trypsin digested (15 mg) for 30 minutes at 30 C in 20 ml PIPES saline. Tissue was washed three times in PIPES saline and triturated in 0.5 ml PIPES saline with Pasteur pipettes of decreasing apertures to dissociate neurons. Neurons were then diluted in 8 ml Dulbecco's modified Eagle's medium with 25 mM HEPES, 25 mM glucose (Gibco), 5% horse serum and placed on 10 mm glass cover slips (poly-D-lysine/laminin, BD Biosciences) in 24 well plates at 37 C. Neurons were studied in voltage-clamp and current-clamp modes within 1 to 5 hours.

Neurons were superfused in bath solutions containing (in mM) 145 NaCl, 5.4 KCl, 2 CaCl2, 1 MgCl2, 10 HEPES, 10 MES and pH was adjusted with TMA·OH. Pipettes (3-5 MΩ polished glass pipettes (Drummond Scientific, 100 μl)) contained (in mM) 5 NaCl, 90 K-gluconate, 15 KCl, 1 MgCl2, 10 EGTA, 60 HEPES, and 3 Na2ATP, adjusted to pH 7.3 with KOH. Extracellular pH was switched with a Rapid Solution Changer (RSC-200; Biologic, Grenoble, France). In voltage-clamp mode, membrane potential was maintained at −70 mV. In current clamp mode, holding voltage was adjusted to −77+/−2 mV. Inhibitory neurons were identified by location (lacunosum moleculare layer microdissection), round morphology, and size (4.4+/−0.3 um). Excitatory, pyramidal neurons were identified by location (CA1 microdissection), pyramidal morphology, spike frequency adaptation in response to current injection, and size (8.3+/−0.3 um).

### Example 5

Applicants injected ASIC1a+/+WT and ASIC1a−/− mice with kainate, a chemoconvulsant that activates glutamate receptors, and assessed seizure severity. During the first 20 minutes after injection, mice with both genotypes had similar seizures affecting the head or forelimbs (FIG. 1a). However with time, the ASIC1a-null mice developed more severe seizures. FIG. 1 demonstrates that ASIC1a reduces seizure severity. Seizure response over time varies between ASIC1a+/+ (+/+) and ASIC1a−/− mice (−/−) following 20 mg/kg IP kainate (+/+, n=6; −/− n=7) (FIG. 1a). For each ten-minute interval, the highest level of seizure activity was scored using the Racine seizure scale. ANOVA with repeated measures revealed a significant main effect of time (df(1,6), F=27.3, p<0.0001) and a significant time×genotype interaction (df(1,6), F=2.39, p=0.039), illustrating that as time passes seizures become more severe in the ASIC1a−/− mice. The maximum Racine score during the 60 minute trial was measured (Mann-Whitney U test; **p=0.004) and are depicted in FIG. 1b.

PTZ (thought to cause seizures by inhibiting multiple targets, including GABA receptors) was also administered to quantify the percentage of mice that developed GTCS. The majority of ASIC1a−/− mice developed GTCS, whereas WT mice were less likely to have GTCS. FIG. 1c shows the incidence of generalized GTCS in ASIC1a+/+ and ASIC1a −/− mice following 50 mg/kg IP PTZ (+/+, n=12; −/−, n=8; Fisher's exact test; **p=0.004). Thus with two different chemoconvulsants, loss of ASIC1a increased seizure severity.

### Example 6

ASIC1a was also acutely inhibited in wild-type mice with an intracerebroventricular (ICV) injection of the ASIC1a antagonist psalmotoxin 1 (PcTx1), blocking ASIC1a effects on ischemic stroke and fear. PcTx1 increased the incidence of continuous GTCS following kainate injection (FIG. 1d). Similar effects on seizure activity with both ASIC1a gene disruption and pharmalogical blockade indicate that developmental abnormalities were not responsible for the effects in ASIC1a−/− mice.

Left-lateral, ICV guide cannulae were implanted in anesthetized mice (relative to bregma: anteroposterior −0.3 mm, lateral−1.0 mm, ventral=3.0 mm) and verified by methylene blue injection after euthanasia. Three to five days later, 5 uL or PcTx1-containing venom (SpiderPharm, Yarnell, Ariz.) (9 mg/uL) was injected in ACSF (in mM: NaCl 124, KCl 3, NaH2PO4 1.2, MgSO4 1.2, CaCl2 2, NaHCO3 26) or ACSF alone into wild-type mice using a 10 uL-Hamilton syringe connected to a 30-gauge injection (over 10 seconds). Two hours later, mice were injected with kainate (20 mg/kg, IP) then mouse behavior was scored. Continuous, tonic-clonic seizures were identified by tonus and clonus in all four limbs with loss of posture lasting greater than 60 seconds.

### Example 7

Kainate or PTZ was injected into a mouse, utilizing higher doses to increase the chances of identifying a protective effect compared to WT littermates. Transgenic mice over-expressing ASIC1a via a pan-neuronal synapsin 1 promoter (ASIC1aTg+) showed that the ASIC1a expression was increased throughout the brain, and the neurons had larger amplitude acid-evoked currents than WT littermates. ASIC1a over-expression reduced the incidence of GTCS after PTZ injection (FIG. 1g) and reduced seizure severity following kainate injection (FIG. 1e, FIG. 1f).

The incidence of GTCS in ASIC1a+/+ (WT) and ASIC1aTg+ mice following 30 mg/kg IP kainate (WT, n=13; Tg+, n=10; Fisher's exact test #p=0.06) demonstrates the relationship between the level of ASIC1a expression and the degree of seizure protection (FIG. 1f, FIG. 1g). The incidence of GTCS in WT and Tg+ mice following 65 mg/kg IP PTZ (WT, n=13; Tg+, n=13; Fisher's exact test *p=0.037) demonstrates the same relationship between the level of ASIC1a expression and the degree of seizure protection using of a different convulsant (FIG. 1g).

### Example 8

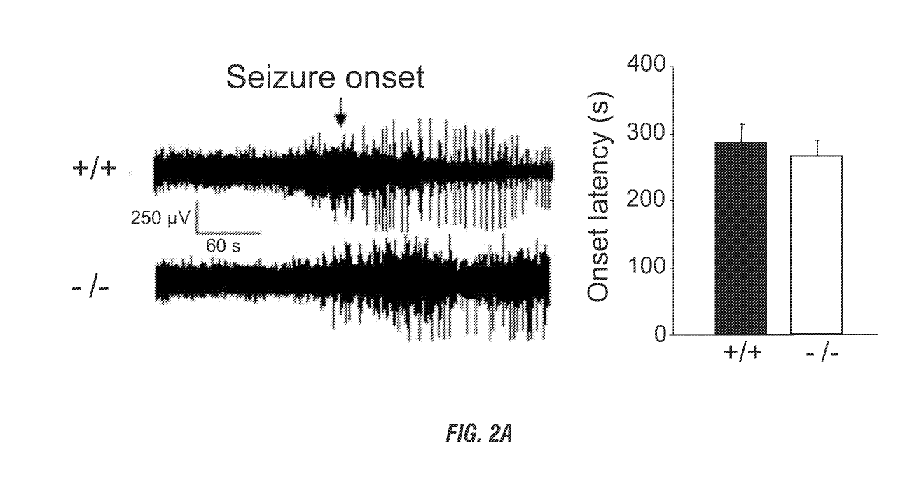

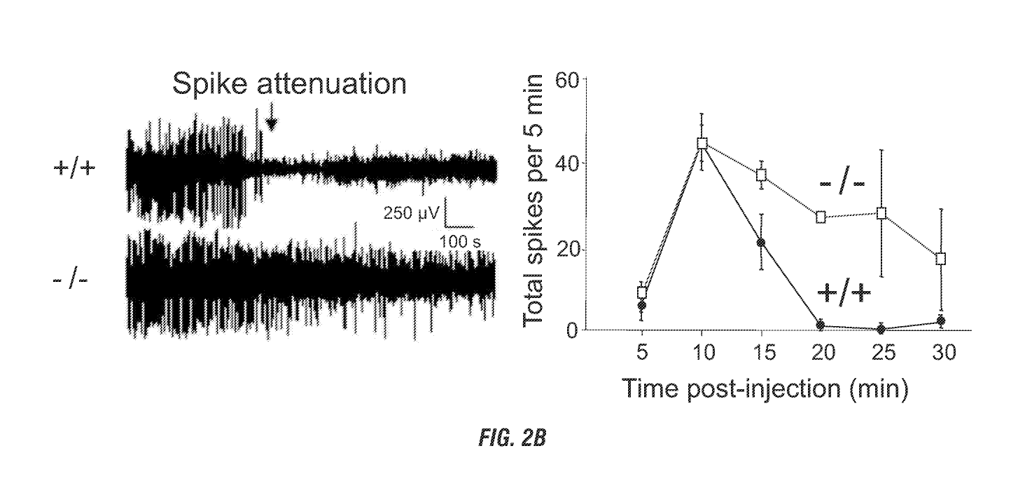

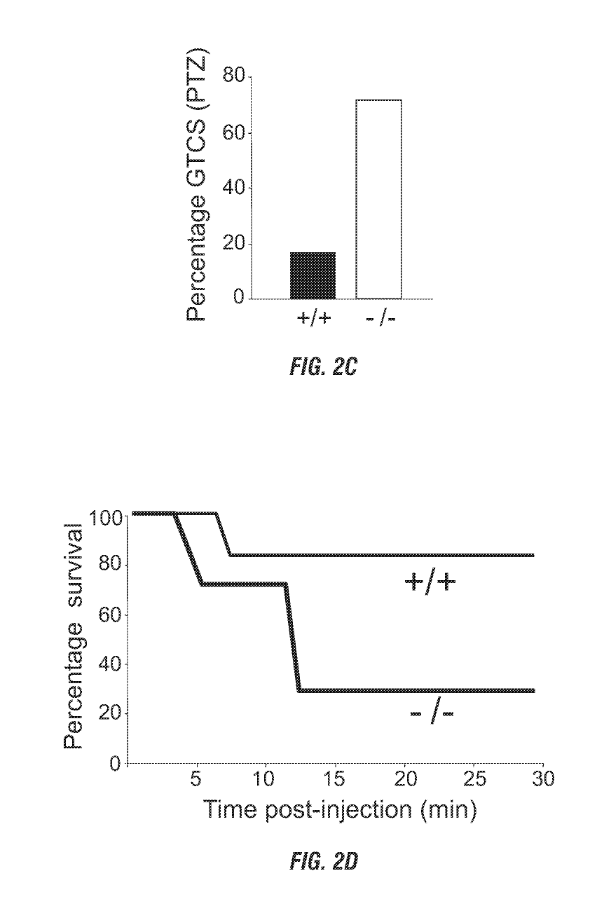

The representative EEG tracings and quantification of time from IP injection of PTZ (50 mg/kg) until first seizure spikes in ASIC1a+/+ and ASIC1a−/− mice was evaluated (+/+, n=6; −/−, n=7; unpaired T-test: df (11), t=0.544, p=0.597). FIGS. 2a-2c demonstrate that ASIC1a disruption increases seizure duration and progression, but does not influence seizure onset. The latency to EEG spike activity was the same in ASIC1a−/− and WT mice (FIG. 2a). In addition, within the first 10 minutes following PTZ injection, both WT and ASIC1a−/− mice had seizures of similar severity characterized by myoclonic jerks and a similar number of EEG spike discharges (FIG. 2b).

As the seizures continued, differences between the genotypes became apparent. In WT mice, EEG spikes decreased precipitously following the 10-minute time point (FIG. 2b), whereas most ASIC1a-null mice continued to have seizure spikes even after 20 minutes; this seizure activity often progressed to tonic-clonic seizures and death (FIG. 2c, FIG. 2d). Surviving ASIC1a−/− mice continued to have more seizure activity than wild-type mice (FIG. 2b). However, only EEG spike activity in surviving animals could be measured, which likely underestimates the deficit in seizure termination in ASIC1a−/− mice.

Representative EEG tracings and total number of seizure spikes per five minute interval in surviving mice (+/+, n=6; −/−, n=7) are depicted in FIG. 2b. The spike numbers varied significantly with time (Mixed model analysis; df (1, 5), F=23.5, p<0.001), and there was a significant time×genotype interaction (df (1,6), F=32.9, p<0.001), showing that ASIC1a−/− mice had prolonged seizure activity as time elapsed. Furthermore, the incidence of GTCS increases and the percent survival decreases with increasing ASIC1a disruption (+/+, n=6; −/−, n=7; Fisher's exact test; #p=0.078) (FIG. 2c). The percentage of survival over time (+/+, n=6; −/−, n=7; Mantel-Cox Log Rank, p=0.025) is further depicted in FIG. 2d.

### Example 9

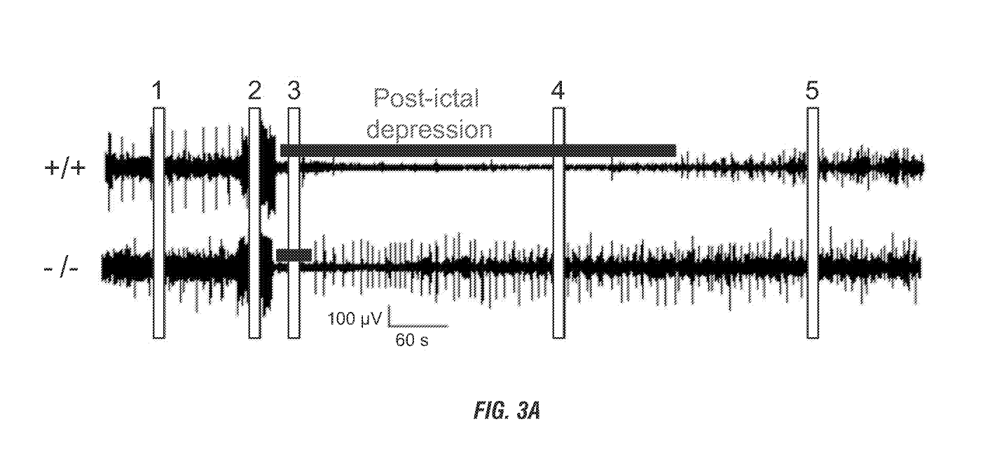

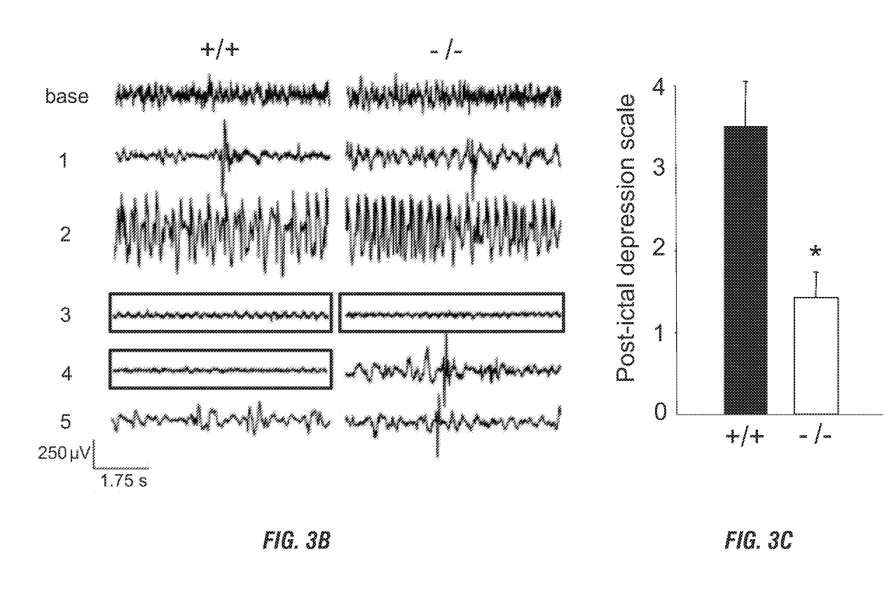

In WT mice, seizures were often followed by a suppression of spike discharges. This low-amplitude EEG pattern, called post-ictal depression, has been suggested to result from the factors that cause seizure termination. Representative EEG tracings from an ASIC1a+/+ and ASIC1a−/− mouse approximately 5 minutes following IP injection of PTZ (50 mg/kg) are shown in FIG. 3a. Five, 5-second intervals are denoted by vertical bars (FIG. 3a, labeled 1 through 5). These same intervals are shown in FIG. 3b in rows using an expanded time scale and showing EEG tracings prior to PTZ injection (base), initial spike wave activity (1), and seizures associated with forelimb clonus (2) were similar in both genotypes. Immediately following seizures (3), mice entered a period of post-ictal depression, shown by horizontal bars (FIG. 3a, labeled post-ictal depression). Post-ictal depression quickly reverted to seizure activity in ASIC1a−/− mice (4, 5).

In contrast to WT mice, ASIC1a−/− mice had only brief periods of EEG depression that were interrupted by seizure spikes (FIG. 3a, FIG. 3b). ASIC1a disruption significantly reduced post-ictal depression. The quantification of post-ictal depression as scored using the post-ictal depression scale (+/+, n=6; −/−, n=7; Mann-Whitney U test, *p=0.011) is denoted in FIG. 3c. This loss of an EEG pattern associated with seizure termination is consistent with the prolonged seizure activity and the increased severity observed in ASIC1a−/− mice.

Post-ictal depression was defined as a low-amplitude, slow-wave EEG signal without seizure spikes occurring after a seizure. The duration of post-ictal depression was defined from its onset following a seizure until the resumption of seizure spikes or return of the EEG signal to an amplitude exceeding 2 mV. Based on seizure severity and the longest observed period of post-ictal suppression, each mouse was scored and separated into one of five categories: (1) no post-ictal depression and lethal seizures, (2) no post-ictal depression with persistent seizure activity, (3) depression<60 seconds, (4) depression 60-180 seconds, (5) depression>180 seconds.

### Example 10

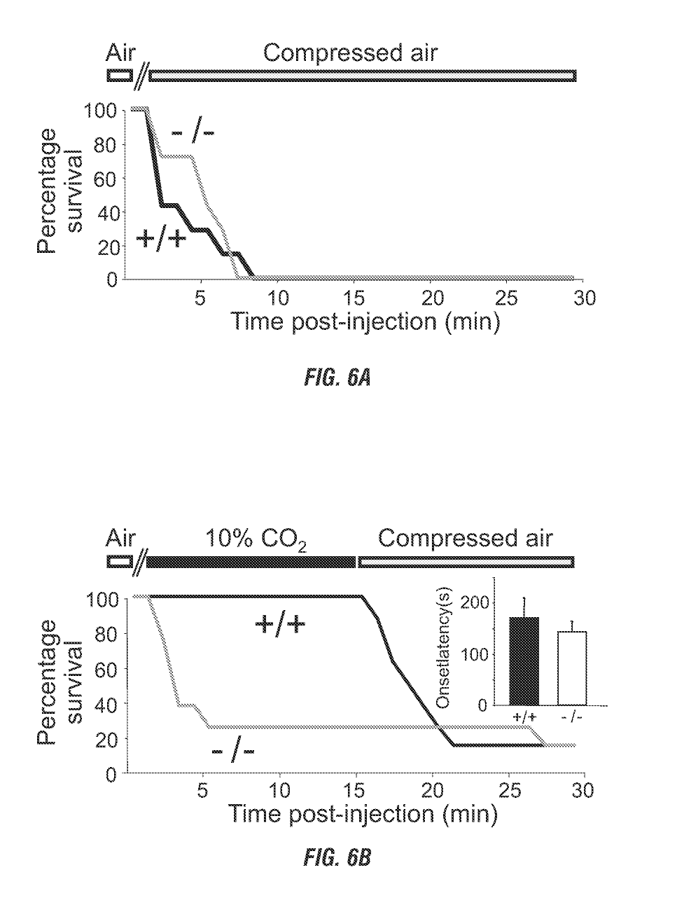

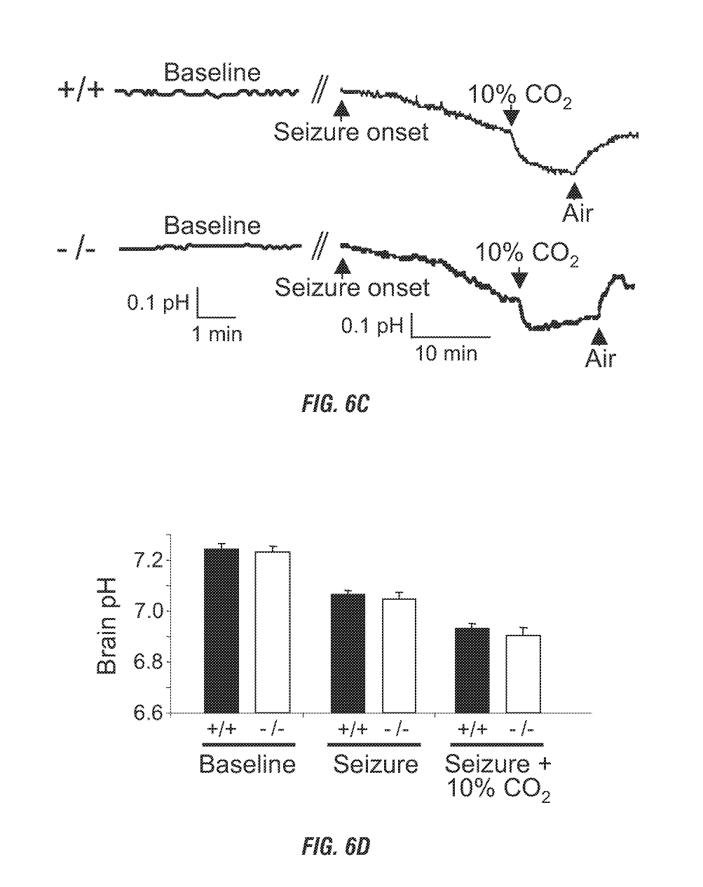

Age (13-16 weeks) and gender-matched ASIC1a+/+ and ASIC1a−/− mice were injected IP with 90 mg/kg PTZ to test the anti-epileptic effects of CO₂. A high dose of PTZ was administered to evoke lethal seizures in mice of both genotypes (FIG. 6a). Kaplan-Meier survival analysis of ASIC1a+/+ and ASIC1a−/− mice in response to 90 mg/kg PTZ while breathing compressed air was conducted (+/+, n 7; −/−, n=7). Both mice had the same rate of reduced survival (Mantel-Cox Log Rank, p=0.582).

After the onset of generalized-clonic seizures (identified behaviorally by clonus in all four limbs), compressed air or 10% CO₂ (in air) was rapidly administered in an airtight Plexiglas chamber for 15 minutes (FIG. 6b). The onset latency and time of CO₂ administration was similar between genotypes (inset) (unpaired T-test, df (14), t=0.663, p=0.518). The chamber was perfused with CO₂ until minute 15, and then switched to compressed air for the duration of the trial (+/+, n=8; −/−, n=8). In CO₂, the likelihood of survival was significantly greater in the ASIC1a+/+ mice (Mantel-Cox Log Rank, p=0.002). The administration of the 10% CO₂ prevented lethal seizures in WT mice, but had little effect in ASIC1a−/− mice. In the ASIC1a−/− mice, seizures continued to progress rapidly to death. All of the ASIC1a+/+ mice survived until the CO₂ was switched back to air at minute 15, whereupon the mice rapidly died. The results verified the in vitro findings and taught that ASIC1a also mediates the anti-epileptic effects of low pH in vivo.

To verify brain pH drops in vivo during seizures and CO2 inhalation, a fiber optic pH sensor (pHOptica, Sarasota, Fla.) was implanted into the lateral cerebral ventricle of wild-type and ASIC1a−/− mice. Age (13-15 weeks) and gender-matched ASIC1a+/+ and ASIC1a−/− mice were anesthetized with ketamine/xylazine. Sixty minutes after sedation, the fiber optic pH sensor was placed in the left lateral ventricle. The sensor was calibrated at 35 C and pH values were calculated using pHOptica-v1.0 software (inputting the mouse core temperature under anesthesia). After five minutes of baseline pH measurement, PTZ was injected IP. Due to the anesthesia, a high dose (120 mg/kg) of convulsant was required to achieve an approximate level of seizure activity seen in unanesthetized mice. If generalized seizures did not occur, additional 60 mg/kg PTZ was injected every 20 minutes until seizures began. The total amount of convulsants administered did not differ between the genotypes (ASIC1a+/+=252+/−34.9 mg/kg, ASIC1a−/−=216+/−14.7 mg/kg).

Generalized seizures caused brain pH to fall (pH approximately 7.05) (FIG. 6c, FIG. 6d). CO2 inhalation rapidly and reversibly lowered pH even further in seizing mice (pH approximately 6.9). Such pH levels elicit robust ASIC1a currents and firing of inhibitory neurons. Brain pH fell to similar levels in mice of both genotypes, indicating ASIC1a also mediates the antiepileptic effects of low pH in vivo.

We claim:

1. A method of treatment and prevention of a patient for seizures or a seizure disorder, comprising administering to a patient in need thereof a therapeutically effective amount of an ASIC1a receptor activator and a pharmaceutically acceptable carrier.

2. The method of claim 1 wherein the ASIC1a receptor activator is selected from the group consisting of ASIC1a agonists, prodrugs, receptor modulators, activity enhancers, and combinations of the same.

3. The method of claim 1 wherein the seizure or seizure disorder is selected from the group consisting of absence seizures, atonic seizures, tonic-clonic seizures, myoclonic seizures, simple partial seizures, complex partial seizures, nonepileptic seizures, and status epilepticus seizures.

4. The method of claim 2 wherein the ASIC1a receptor activator and pharmaceutically acceptable carrier are administered by a route selected from the group consisting of orally, intravenously, intramuscularly, topically, sublingually, buccally, intranasally, and rectally.

5. The method of claim 2 wherein the ASIC1a receptor activator and carrier treatment are administered to reduce the severity of the seizure.

6. The method of claim 2 wherein the ASIC1a receptor activator and carrier treatment are administered to prevent the progression of a seizure.

7. The method of claim 2 wherein the ASIC1a receptor activator and carrier treatment are administered to prevent the onset of status epilepticus seizures.

8. The method of claim 2 wherein the ASIC1a receptor activator and carrier treatment are administered to decrease the incidence of tonic-clonic seizures.

9. The method of claim 2 wherein the ASIC1a receptor activator and carrier treatment are administered to increase post-ictal depression associated with seizure termination.

10. The method of claim 2 wherein the ASIC1a receptor activator and carrier treatment are administered to raise action potential threshold associated with seizure termination.

11. The method of claim 2 wherein the ASIC1a receptor activator mediates the central nervous system's response to acidosis.

12. A method of terminating seizures in a patient, comprising administering to a patient in need thereof a therapeutically effective amount of carbon dioxide, an ASIC1a receptor activator and a pharmaceutically acceptable carrier.

13. A method of treatment and prevention for seizures or a seizure disorder comprising activating the ASIC1a channel in a patient in need thereof.

14. The method of claim 13 further comprising stimulating action potential firing in inhibitory neurons to treat and prevent seizures.

15. The method of claim 13 wherein the ASIC1a channel is activated by an ASIC1a receptor activator selected from the group consisting of an ASIC1a agonist, prodrug, receptor modulator, activity enhancer, and combinations of the same.

16. A pharmaceutical composition for treatment and prevention of seizures comprising a therapeutically effective amount of an ASIC1a receptor activator and a pharmaceutically acceptable carrier.

17. The composition of claims 14 wherein the composition is formulated to be administered by a route selected from the group consisting of orally, intravenously, intramuscularly, topically, sublingually, buccally, intranasally, and rectally.

18. The composition of claim 14 wherein the ASIC1a receptor activator is selected from the group consisting of ASIC1a agonists, prodrugs, receptor modulators, activity enhancers, and combinations of the same.

19. The composition of claim 14 wherein the pharmaceutically acceptable carrier is selected from the group consisting of a carrier, diluent, excipient, wetting agent, buffering agent, suspending agent, lubricating agent, adjuvant, vehicle, delivery system, emulsifier, disintegrant, absorbent, preservative, surfactant, and combinations of the same suitable for use in the composition.

20. A method of making a pharmaceutical composition for treatment and prevention of seizures or seizure disorders comprising:
synthesizing an ASIC1a receptor activator; and
combining said ASIC1a receptor activator with a pharmaceutically acceptable carrier.

21. A method for identifying a compound for treating or preventing seizures, comprising:
administering a compound to be screened to cells;
expressing ASIC1a in the presence of acid and the compound; and
determining whether the compound activates or enhances the activity of ASIC1a channels.
